(19) United States
(12) Patent Application Publication
(10) Pub. No.: US 2026/0345547 A1
(43) Pub. Date: Nov. 5, 2026

(54) USE OF DIHYDROQUERCETIN IN TREATING OCULAR SURFACE DISEASES

(71) Applicant: HE UNIVERSITY SCHOOL OF CLINICAL MEDICINE, Liaoning (CN)

(72) Inventors: Donglei ZHANG, Liaoning (CN); Zhenli YUAN, Liaoning (CN); Yuanlong AN, Liaoning (CN); Minnan WANG, Liaoning (CN); Qi GONG, Liaoning (CN); Xiangdong HE, Liaoning (CN); Wei HE, Liaoning (CN)

(21) Appl. No.: 19/114,110

(22) PCT Filed: Nov. 14, 2023
(86) PCT No.: PCT/CN2023/131428

§ 371 Date: Mar. 21, 2025

(30) Foreign Application Priority Data
Dec. 2, 2022 (CN) 202211536112.X

(51) Int. Cl. A61K 31/353; A61K 9/00; A61P 27/02

(52) U.S. Cl. CPC A61K 31/353; A61K 9/0048; A61P 27/02

(57) ABSTRACT

Disclosed is use of dihydroquercetin in treating ocular surface diseases, pertaining to the technical field of pharmacy. The use of dihydroquercetin in treating ocular surface diseases of the present invention is specifically use of dihydroquercetin in preparing a medicament for preventing, alleviating and/or treating ocular surface diseases. The present invention applies dihydroquercetin to the prevention, alleviation and/or treatment of ocular surface diseases for the first time. The dihydroquercetin serving as an antioxidant has a good protective effect against diseases related to oxidative damage in human corneal epithelial (HCE) cells. Moreover, in a certain concentration range, the dihydroquercetin can promote the proliferation and improve the vitality of the HCE cells, and can also increase the tear secretion amount and tear film stability of the HCE cells, resulting in significant amelioration of the ocular surface diseases.

### RELATED APPLICATIONS

This application is a 35 U.S.C. § 371 filing of International Patent Application No. PCT/CN2023/131428, filed Nov. 14, 2023, which claims priority to Chinese Patent Application No. 202211536112.X, filed Dec. 2, 2022, the entire disclosures of which are hereby incorporated by reference in their entirety.

### FIELD

The present disclosure relates to the technical field of pharmaceuticals, and in particular to use of dihydroquercetin in treating ocular surface diseases.

### BACKGROUND

Oxidative damage is prevalent in various diseases and plays a significant role particularly in aging and inflammation-related conditions. Free radicals generated during oxidation can directly affect cell membranes, leading to their disruption. These radicals can also enter cells through membrane channels, causing damage to intracellular proteins and DNA. Oxygen radicals are involved in the metabolism of arachidonic acid, which is crucial in the inflammatory response. Lipid peroxides produced in this process act as chemokines, exacerbating inflammation. Moreover, oxidation products can trigger the production of chemokines different from arachidonic acid, inactivate protein hydrolase inhibitors, and elevate levels of collagenase, leading to the destruction of connective tissue. Common ocular disorders associated with oxidative damage include dry eye and corneal alkali burns.

Dry eye is a common condition in ophthalmology characterized by insufficient tears or excessive tear evaporation, leading to instability of the tear film and ocular surface inflammation. Recent research both domestically and internationally has deepened the understanding of dry eye's pathogenesis. It is now recognized that the disease's development is closely linked to immune-inflammatory responses, apoptosis, and sex hormonal levels, with T cell-mediated immune inflammation being a critical factor. Additionally, high osmotic pressure in the tears, resulting from decreased tear secretion or increased evaporation, plays a crucial role in perpetuating the dry eye symptoms. This high osmotic pressure can induce morphological changes such as apoptosis of conjunctival epithelial cells and a reduction in functional goblet cells, triggering an inflammatory cascade that leads to the death of corneal epithelial cells. The loss of these cells diminishes the mucin and lipid content in tears, exacerbating tear film instability and perpetuating a vicious cycle. Dry eye can cause symptoms such as eye dryness, foreign body sensation, photophobia, and pain. In severe cases, it can lead to corneal damage and threaten visual function. Therefore, urgent clinical research and treatment development for dry eye are essential.

Corneal alkali burns, a prevalent form of ocular trauma with a high risk of blindness, are a significant clinical concern. In the early stages of an alkali burn, apoptosis and the activation of inflammatory responses are key factors contributing to corneal injury. In the later stages, corneal neovascularization can severely impair corneal transparency and visual acuity. However, choroidal neovascularization (CNV) in the fundus is a complex pathological process regulated by multiple factors. A network of growth factors and cytokines precisely controls the formation of CNV. Vascular endothelial growth factor (VEGF) is the most potent pro-angiogenic factor identified, and its overexpression is closely linked to the development of CNV. While various drugs are available to inhibit neovascularization, many have confirmed adverse effects during treatment. For example, common side effects of glucocorticoids include Cushing's syndrome, infections, gastrointestinal issues, edema, glucose metabolism disorders, electrolyte imbalances, and nervous system disturbances. Similarly, anti-VEGF drugs can cause side effects such as conjunctival congestion, ocular pain, foreign body sensation, corneal abrasion, corneal edema, increased intraocular pressure, shadow spreading, intraocular infection, retinal detachment, and vitreous hemorrhage.

Therefore, it is crucial to research and develop novel antioxidants that offer protective effects against diseases associated with ocular oxidative damage.

### SUMMARY

In view of this, the technical problem to be solved by the present disclosure is to provide use of dihydroquercetin in the treatment of an ocular surface disease. The dihydroquercetin has a protective effect against H₂O₂-induced oxidative stress damage in human corneal epithelial (HCE) cells.

In order to achieve the above objects, the following technical solution is applied in the present disclosure.

The present disclosure provides use of dihydroquercetin in the manufacture of a medicament for the prevention, mitigation and/or treatment of an ocular surface disease.

Dihydroquercetin (DHQ), also known as taxifolin, is found in various plants, with high concentrations in larch, especially in Douglas fir. Dihydroquercetin was first extracted and isolated by the Japanese scholar Fukui from the leaves of the coniferous plant Chamaecyparis obtusa. More recently, dihydroquercetin has also been identified in several fruits, including grapes, oranges, and grapefruit. Research indicates that dihydroquercetin is rich in phenolic hydroxyl groups and exhibits a broad range of biological activities. It can both inhibit and activate various enzymes, leading to diverse physiological effects. The chemical structure of dihydroquercetin is represented by the following formula:

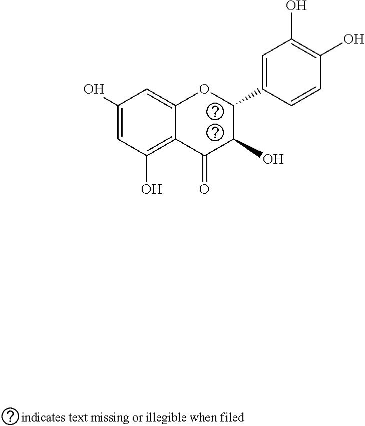 [SMILES: O=C1c2c(O)cc(O)cc2O[C@H](c2ccc(O)c(O)c2)[C@H]1O]

Dihydroquercetin is used for preventing, alleviating and/or treating an ocular surface disease for the first time in the present disclosure.

Preferably, the ocular surface disease is an ocular surface disease caused by corneal epithelial cell damage and/or apoptosis.

Preferably, the corneal epithelial cell damage is oxidative damage.

Preferably, the oxidative damage is caused by hydrogen peroxide.

The dihydroquercetin of the present disclosure has a protective effect against H₂O₂-induced oxidative stress damage in human corneal epithelial (HCE) cells.

The protective effect is specifically that dihydroquercetin within a certain concentration range has a proliferative effect on HCE cells, and can increase the relative cell viability of HCE cells after oxidative damage.

The present disclosure involves the establishment of a model for H₂O₂-induced damage in human corneal epithelial (HCE) cells. The concentration of H₂O₂ required for effective modeling was determined, and this concentration was then used to induce oxidative damage in the HCE cells. Subsequently, different concentrations of dihydroquercetin were applied to the HCE cells. It was found that a certain concentration of dihydroquercetin promoted HCE cell proliferation, while excessively high concentrations were toxic to the HCE cells.

Specifically, the concentration for modeling is 300 μM. In some specific embodiments of the present disclosure, the concentration of dihydroquercetin that has a proliferation effect on HCE cells is 25-200 μM, and the concentration of dihydroquercetin that can produce a certain degree of toxicity to HCE cells is 600 μM.

Preferably in the present disclosure, the ocular surface disease is dry eye or a disease related to corneal neovascularization.

The experimental results demonstrate that treatment with dihydroquercetin increased tear secretion and tear film stability of HCE cells to a certain extent, and significantly improved dry eye.

The present disclosure further provides a dihydroquercetin ophthalmic preparation comprising dihydroquercetin and a solvent.

Preferably, the dihydroquercetin has a mass concentration of 0.01-0.5%; more preferably, the dihydroquercetin has a mass concentration of 0.03-0.3%; further preferably, the dihydroquercetin has a mass concentration of 0.03% or 0.3%.

Preferably, the dihydroquercetin ophthalmic preparation is in a dosage form selected from the group consisting of eye drops, ophthalmic ointment, periocular and intraocular injection, ophthalmic gel and liposome.

In contrast to the prior art, the present disclosure provides use of dihydroquercetin in the manufacture of a medicament for the prevention, alleviation and/or treatment of an ocular surface disease. Dihydroquercetin is used for preventing, mitigating and/or treating an ocular surface disease for the first time in the present disclosure. The dihydroquercetin has a protective effect as an antioxidant against a disease related to oxidative damage in human corneal epithelial (HCE) cells. Moreover, when used within a specific concentration range, dihydroquercetin promotes HCE cell proliferation, enhances cell viability, and increases tear secretion and tear film stability, leading to significant improvement in ocular surface diseases.

### BRIEF DESCRIPTION OF DRAWINGS

FIG. 1 shows the results of the MTS assay conducted after 24 hours of treatment on corneal epithelial cells with different concentrations of dihydroquercetin (A), different concentrations of H₂O₂ (B), and combinations of 300 μM hydrogen peroxide and different concentrations of dihydroquercetin (C), where P indicates the significant level, ** represents P<0.01, *** represents P<0.001, ##represents P<0.01, and ####represents P<0.001.

FIG. 2 shows the results of tear secretion amount measurements from live mice in animal experiments.

FIG. 3 shows the results of tear film breakup time measurements from live mice in animal experiments.

FIG. 4 shows the corneal morphology of mice in each experimental group after sodium fluorescein staining of cornea.

FIG. 5 shows the scoring results of corneal epithelial damage in mice examined by sodium fluorescein staining of cornea.

FIG. 6 shows the morphology of corneal neovascularization in New Zealand rabbits from each experimental group observed using a slit lamp for corneal.

FIG. 7 show the results of neovascularization area of the corneal of New Zealand rabbits in each experimental group, where **** represents P<0.0001.

### DETAILED DESCRIPTION

To further illustrate the present disclosure, the use of dihydroquercetin in the treatment of an ocular surface disease provided by the present disclosure is described in detail below in conjunction with embodiments.

Human corneal epithelial cells were purchased from the American Type Culture Collection (ATCC). The cell medium used was high-sugar DMEM medium supplemented with 10% (V/V) fetal bovine serum, 100 U/mL penicillin, and 100 μg/mL gentamicin. The cells were cultured in an incubator containing 5% carbon dioxide at 37° C. Cells in the logarithmic growth phase were seeded into a 96-well plate at a density of 1.5×10⁵ cells/mL, with 100 μL per well and 6 replicate wells per group. The cells were grouped into a normal cell group, a hydrogen peroxide model group, and intervention groups treated with dihydroquercetin at various concentrations. The assay was performed using an MTS kit (e.g., Promega), and absorbance was measured at 490 nm using a microplate reader.

Survival rate=survival rate of each group/survival rate of normal cell group×100%

### Example 1

### I. MTS Assay

Human corneal epithelial cells were purchased from the American Type Culture Collection (ATCC). The cell medium used was high-sugar DMEM medium supplemented with 10% (V/V) fetal bovine serum, 100 U/mL penicillin, and 100 μg/mL gentamicin. The cells were cultured in an incubator containing 5% carbon dioxide at 37° C. Cells in the logarithmic growth phase were seeded into a 96-well plate at a density of 1.5×10⁵ cells/mL, with 100 μL per well and 6 replicate wells per group. After 24 h, the cells were treated with H₂O₂ or dihydroquercetin according to the experimental purpose. For color development, 20 μL of MTS solution was added to each well, and the cells were incubated for another 2-4 h. To measure the colorimetric results, the absorbance value of each well was read at the wavelength of 490 nm using an enzyme-linked immunosorbent assay reader. The results were recorded, and a cell growth curve was plotted with time as the abscissa and absorbance value as the ordinate.

II. Establishment of H₂O₂-Induced Human Corneal Epithelial (HCE) Cell Damage Model

HCE cells were subcultured and seeded at 1.5×10⁵ cells/mL, with 100 μL per well. Various concentrations of H₂O₂ (100 μM, 200 μM, 300 Mm, 400 μM, 500 μM, 600 μM, 700 μM, 800 μM, and 900 μM) were applied to the HCE cells for 24 h. Six replicate wells were set for each group to ensure the reliability and accuracy of the experimental data, and the degree of cell damage by H₂O₂ was observed. For color development, 20 μL of MTS solution was added to each well, and the cells were incubated for another 2-4 h. To measure the colorimetric results, the absorbance value of each well was read at the wavelength of 490 nm using an enzyme-linked immunosorbent assay reader. The results were recorded, and a cell growth curve was plotted with various concentrations of H₂O₂ as the abscissa and the absorbance value as the ordinate. The concentration of H₂O₂ that resulted in approximately 50% cell viability, as determined by the MTT assay, was used as the modeling concentration for the oxidative damage model. Survival rate=survival rate of each group/survival rate of normal cell group×100%.

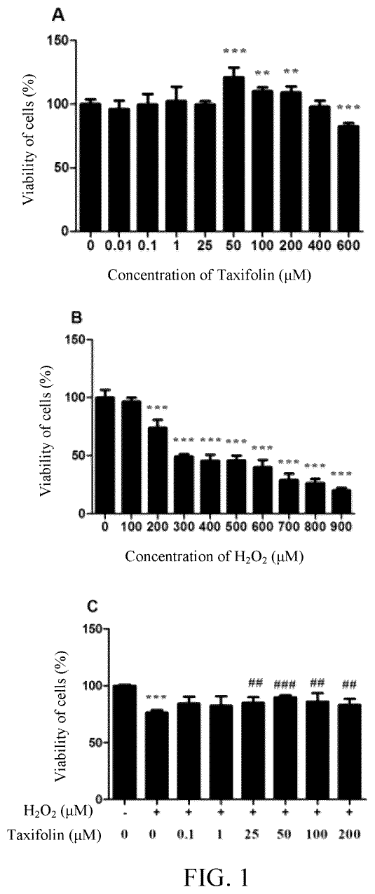

FIG. 1 shows the results of the MTS assay conducted after 24 hours of treatment on corneal epithelial cells with different concentrations of dihydroquercetin (A), different concentrations of H₂O₂ (B), and combinations of 300 μM hydrogen peroxide and different concentrations of dihydroquercetin (C), where P indicates the significant level, ** represents P<0.01, *** represents P<0.001, ##represents P<0.01, and ####represents P<0.001. As shown in FIG. 1B, the cell survival rate was 49% when the H₂O₂ concentration was at 300 μM, so 300 μM H₂O₂ was chosen as the modeling concentration for the oxidative damage model in subsequent experiments.

### (3) Proliferative and Toxic Effects of Dihydroquercetin on HCE Cells

Various concentrations of dihydroquercetin (0.01-600 μM) were applied to HCE cells, and 6 replicate wells were set for each group to ensure the reliability and accuracy of the experimental data. The proliferative effect of the drug on the cells was then assessed.

As shown in FIG. 1A, after 24 h of treatment on HCE cells with different concentrations of dihydroquercetin, the cell viability of HCE cells in the experimental groups with concentrations of 50 μM, 100 μM, and 200 μM was significantly higher compared to the control group, indicating that dihydroquercetin at a concentration of 50-200 μM had an effect of promoting proliferation on HCE cells. When the dihydroquercetin concentration was increased to 600 μM, the cell viability of HCE cells was significantly reduced compared to the control group, indicating that dihydroquercetin at a concentration of 600 μM had a toxic effect on HCE cell.

### (4) Anti-Oxidative Damage Effect of Dihydroquercetin on HCE Cells

The experiment included 8 groups: a normal control group, a H₂O₂ model group, and intervention groups treated with various concentrations of dihydroquercetin (0.1-200 μM)+H₂O₂. HCE cells were treated with 300 μM H₂O₂ for modeling, and then the drug was applied on HCE cells at different concentrations within the safe concentration range. The survival rate of cells in each group was assessed by the MTS assay to evaluate the protective effect of dihydroquercetin against oxidative damage of HCE cells.

As shown in FIG. 1C, for the experimental groups, the cell survival rate in the H₂O₂ model group was lower than that of the control group (P<0.001), and the cell survival rate of 25-200 μM dihydroquercetin group was higher that of the H₂O₂ model group (P<0.01), indicating that H₂O₂ can induce oxidative stress damage in HCE cells, and that dihydroquercetin had a protective effect against oxidative stress damage of HCE cells induced by H₂O₂.

### 1.1 Dry eye

### 1.1.1 Grouping

Out of 50 healthy 8-week-old female C57/BL6 mice, 30 were selected for the experiment. Five mice were randomly assigned to each group, resulting in a total of six groups: blank control group (N), model group (M), positive drug group (CsA), low concentration traditional Chinese medicine group (SL), medium concentration traditional Chinese medicine group (SM), and high concentration traditional Chinese medicine group (SH). During grouping, identification was marked on the mice's tails and cage cards, which included details such as the experiment name, experiment number, group designation, administration dosage, and administration schedule.

### 1.1.2 Feeding

The experimental animals were housed in the SPF-grade laboratory at the Animal Center of He University School of Clinical Medicine, Shenyang. Five mice were reared in each cage (40 cm long×20 cm wide×20 cm high) during the experimental period, with a total of 6 cages. The cages were lined with corn cob bedding. The mice were provided with sterilized, complete nutritional feed purchased from Liaoning Changsheng Biotechnology Co., Ltd. Drinking-water was pure water prepared by the HT-RO1000 water purification system in the animal center, and the water quality was tested annually by specialized personnel. During the experimental period, mice were allowed to eat and drink freely. The night before the mice were to be dissected for obtaining material, they were deprived of food but allowed to drink. The temperature and humidity in the animal laboratory were controlled automatically by an air-conditioning unit, maintaining at a temperature of 20-25° C. and a humidity of 40%-70%, and changes in temperature and humidity were recorded daily. The light cycle in the animal laboratory was controlled automatically by a lighting system to ensure a consistent light/dark cycle over a 24-hour period. In the animal laboratory, noise levels were kept to be no higher than 60 decibels. This experiment was approved and supervised by the Institutional Animal Care and Use Committee (IACUC) and adhered strictly to the national animal welfare regulations outlined in the Guiding Opinions on Caring for Animals.

### 1.2 Modeling Method

In this experiment, a mouse model of dry eye was established using an intelligent dry environment control system in combination with scopolamine injection. A two-step dehumidification method was used to control the humidity of the environment during the modeling process. Initially, a temperature-adjustable industrial dehumidifier was used to reduce the room humidity to 40%±5%. Subsequently, an intelligent drying box with a dehumidification range of RH 10%-80% was used to further reduce the humidity of the environment for raising mouse to 15%±3%. At the same time, a noiseless, adjustable-speed (wind speed range of 0-5 m/s) fan was placed inside the intelligent drying box, positioned 20 cm away from the mouse cage at the same vertical height as the mice. These measures ensured effective control of the environmental condition parameters required for the experiment. Mice in the model control group (M), positive drug group (CsA), low concentration dihydroquercetin group (E1), high concentration dihydroquercetin group (E2), and solvent control group were reared in this dry environment (humidity of 15%±3%, wind speed of 2.1±0.2 m/s, and temperature of 21-23° C.), and mice in the blank control group were reared in a normal environment (humidity of 60%-80% and temperature of 21-23° C.). Except for the blank control group, mice in the other groups were injected subcutaneously with 0.5 mg/0.2 mL scopolamine solution three times a day (at 9:00 a.m., 12:00 p.m., and 3:00 p.m.), and the modeling period lasted for 2 weeks. The drug was administered ocularly, with 5 μL drops into the intra-conjunctival sac of each eye twice a day (at 9:00 a.m. and 3:00 p.m.) starting one day before modeling and continued for 15 consecutive days for each treatment group.

### 1.3 Administration Method and Dosage

The drug was administrated ocularly, and all experimental mice were administered starting one day prior to modeling. The blank control group and model control group were not administered. In the positive drug group, cyclosporine A at a mass concentration of 0.05% was instilled into the conjunctival sac as 5 μL drops. In the low concentration treatment group, dihydroquercetin at a mass concentration of 0.03% was administered into the eye as 5 μL drops, while in the high concentration treatment group, dihydroquercetin at a mass concentration of 0.3% was administered into the eye as 5 μL drops. All treatments were applied twice daily at 9:00 a.m. and 3:00 p.m.

### 1.4 Evaluation of Model

On the 14th day of modeling, following the completion of modeling and administration operations, corneal epithelial damage and tear secretion were assessed in the mice from the blank control group and the model control group. Successful modeling was indicated by a significant difference in the indicators between these two groups.

### 1.5 Indicators for Experimental Examination

### 1.5.1 General Observation

During the acclimatization period (prior to grouping), an ophthalmological examination was performed on all mice to detect any visual defects, eye abnormalities, or corneal damage, ensuring that no pre-existing abnormalities were present in the experimental mice. Throughout both the acclimatization period and the experimental process, the mice were weighed weekly. During the administration period, daily observations were conducted before and after treatment to monitor their health status and record any abnormal signs. Observations included: appearance (eyes, ears, mouth, nose, vulva, fur, excreta, limb activity, mental status), signs of vomiting, activity, diet, gait, near-death signs, and mortality.

### 1.5.2 Measurement of Tear Secretion Amount

The mice were restrained without anesthesia. The lower eyelids were gently lifted using soft forceps, and the folded end of a phenol red cotton thread was inserted into the lower conjunctival sac. The lower eyelids were then released to allow them to close naturally, securing the phenol red cotton thread in place. After 1 minute, the thread was removed. The thread was then placed on a printable ruler, and the length of the red-stained portion was measured and recorded. The data were analyzed statistically using GraphPad Prism 8.0 software.

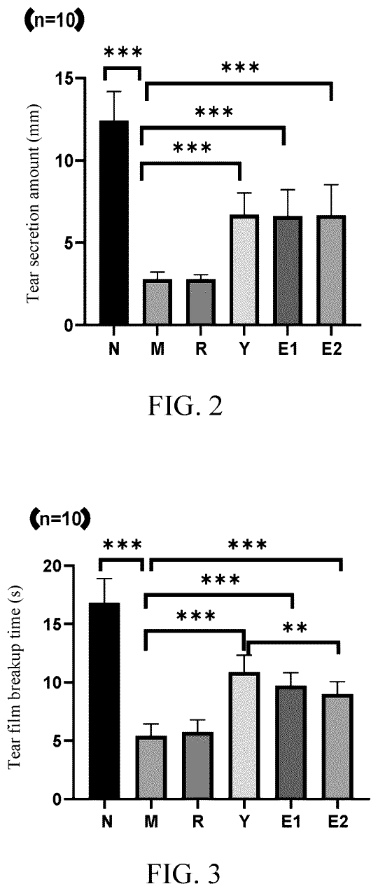

FIG. 2 shows the results of tear secretion amount measurements from live mice in animal experiments. As shown in FIG. 2, N represents the blank control group; M represents the model control group; R represents the solvent control group; Y represents the positive drug group (cyclosporin A at a mass concentration of 0.05%); E1 represents the low concentration dihydroquercetin group; and E2 represents the high concentration dihydroquercetin group. In addition, P denotes a significant difference, with * indicating P<0.05, ** indicating P<0.01, and *** indicating P<0.001.

The length (in mm) of the phenol red cotton thread that changed color was measured. A longer length indicated higher tear secretion, and a shorter length indicated lower tear secretion. Prior to modeling, tear secretion was normal across all groups, with no significant differences between the groups (P>0.05). As shown in FIG. 2, after 14 days of modeling, tear secretion was significantly increased in the positive drug group (CsA), the low concentration dihydroquercetin group (E1), and the high concentration dihydroquercetin group (E2) compared to the model control group (M), with all differences being statistically significant (P<0.001). No significant change in tear secretion was observed in the solvent control group compared to the model control group (M). These results suggest that treatment with dihydroquercetin led to a notable increase in tear secretion in the dry eye model mice.

### 1.5.3 Tear Film Breakup Time Measurement

The mice were restrained without anesthesia, and sodium fluorescein solution was instilled into their conjunctival sacs. After allowing the mice's eyelids to close passively several times, the eyelids were then opened and observed under the cobalt blue light of a slit lamp. The time until the appearance of a block spot due to tear film breakup was recorded. This time is referred to as the tear film breakup time. The data were analyzed statistically using GraphPad Prism 8.0 software.

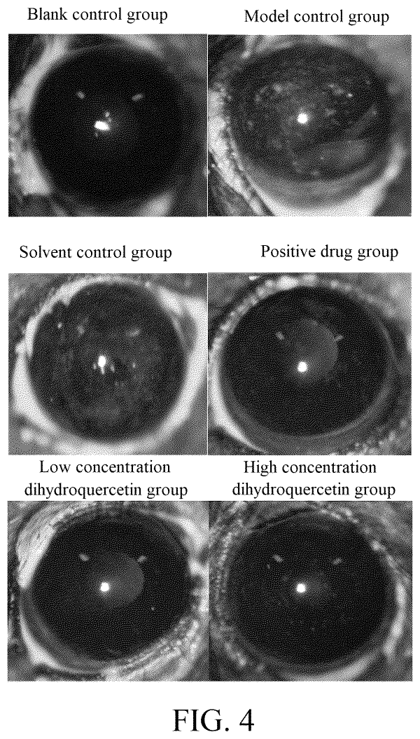

FIG. 3 shows the results of tear film breakup time measurements from live mice in animal experiments. As shown in FIG. 3, N represents the blank control group; M represents the model control group; R represents the solvent control group; Y represents the positive drug group (cyclosporin A at a mass concentration of 0.05%); E1 represents the low concentration dihydroquercetin group; and E2 represents the high concentration dihydroquercetin group. In addition, P denotes a significant difference, with * indicating P<0.05, ** indicating P<0.01, and *** indicating P<0.001. Before modeling, all breakup time (BUT) values were within the normal range, and the differences were not statistically significant (P>0.05). Following modeling with the Intelligent Drying Environment System and drug administration, the differences in BUT values before and after modeling in the blank control group were not statistically significant (P>0.05). After the 14th day (day 14) of modeling, BUT values were measured for all the mice in each group.

The results are shown in FIG. 3: compared to the blank control group, the tear film breakup time of the mice in the other five groups was reduced to varying degrees, with a statistically significant difference (P<0.05); and compared to the model control group, the tear film breakup time of mice in the treatment groups of dihydroquercetin increased significantly, with a statistically significant difference (P<0.05), suggesting that treatment with dihydroquercetin improved the stability of the tear film in the mice.

### 1.5.4 Sodium Fluorescein Staining of Cornea for Assessing Corneal Epithelial Damage

The corneal morphology of experimental mice in each group was observed before modeling and 14 days after modeling. The corneas of both eyes were stained with a sodium fluorescein solution, observed and imaged using a slit lamp. After restraining the mice, 5 μL of 2% sodium fluorescein in saline solution was instilled into the conjunctival sac of the mice. After 1 min, the eyes were gently rinsed with 2 mL of saline, and excess solution was removed with a cotton swab. The sodium fluorescein-stained corneas of the mice were then illuminated with cobalt blue light, observed, imaged and recorded with a slit lamp. Green fluorescent residues on the corneal surface indicated corneal epithelial damage. Sodium fluorescein residues on the corneal surface was scored and recorded. The data were analyzed statistically using GraphPad Prism 8.0 software.

FIG. 4 shows the corneal morphology of mice in each experimental group after sodium fluorescein staining of cornea. As shown in FIG. 4, the corneas of mice in the blank control group were intact and smooth, with no sodium fluorescein staining, indicating their corneal epithelium was undamaged. Except for the blank control group, the model control group and all treatment groups exhibited varying degrees of sodium fluorescein staining, indicating varying degrees of corneal epithelial damage. On the 14th day of modeling, while dense punctate fluorescence was observed in the positive drug group and the dihydroquercetin treatment groups, there was a significant reduction in staining compared to the model control group.

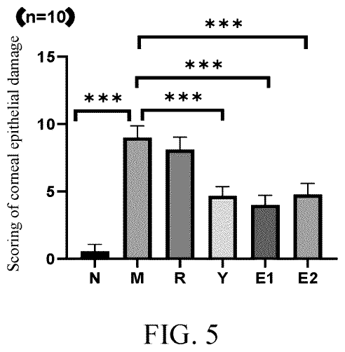

FIG. 5 shows the scoring results of corneal epithelial damage in mice examined by sodium fluorescein staining of cornea, where N represents the blank control group; M represents the model control group; R represents the solvent control group; Y represents the positive drug group (cyclosporine A at a mass concentration of 0.05%); E1 represents the low concentration dihydroquercetin group; and E2 represents the high concentration dihydroquercetin group. In addition, * represents P<0.05, ** represents P<0.01, and *** represents P<0.001. As can be seen from the figure, the corneal epithelium of mice in the model group and in each treatment group showed varying degrees of damage, except for the blank control group. On the 14th day of modeling, corneal epithelial cell damage was also observed in the positive drug group and the dihydroquercetin treatment groups compared to the model control group. However, there was a significant reduction in damage in these groups compared to the model group (P<0.001), indicating that treatment with the positive drug and dihydroquercetin at various concentrations provided a protective effect, reducing the extent of corneal epithelial damage.

2.1 Establishment a Corneal Neovascularization Model with Corneal Alkali Burns

### 2.1.1 Grouping

Fifty 3-month-old healthy New Zealand rabbits, half male and half female, were randomly assigned to six groups, with each group containing six rabbits. The groups were designated as blank control group (N), model control group (M), positive drug group (LEV), low concentration dihydroquercetin group (E1), high concentration dihydroquercetin group (E2) and solvent control group (R). Identification was marked on the rabbits' ears and cage cards, which included details such as the experiment name, experiment number, group designation, administration dosage, and administration schedule.

### 2.1.2 Feeding

Feeding conditions: The experimental animals were housed in the general grade rabbit laboratory at the Animal Center of He University School of Clinical Medicine, Shenyang. During the experimental period, each rabbit was kept in a single stainless steel suspended cage, with a total of six cages used. The animals were allowed to eat and drink freely, and no other species of animals were kept in the same room.

Environmental conditions: The temperature and humidity in the animal room were automatically controlled, with a temperature maintained at 20-25° C. and humidity maintained at 40%-70%. The room was illuminated with artificial light on a 12-hour light/dark cycle, and noise levels were kept below 60 dB.

Animal Welfare: All procedures involving animals adhered to the national animal welfare regulations outlined in the Guiding Opinions on Caring for Animals (2006, Ministry of Science and Technology). The animal use protocol was approved by the Institutional Animal Care and Use Committee (IACUC), which also supervised the experimental process. Humane considerations were prioritized throughout the trial to alleviate any suffering or pain. Euthanasia for humane reasons was performed according to the same procedures as for animals that died during the study. Animals that survived the experiment were euthanized, and their carcasses were disposed of by a professional organization.

### 2.2 Modeling Method

In this experiment, the corneal neovascularization model in rabbits was established using a chemical injury method. The rabbits were generally anesthetized with an intravenous injection of 1% pentobarbital sodium administered via the marginal ear vein. Ocular anesthesia was achieved with proparacaine eye drops. A 6 mm diameter filter paper was soaked in a 1 mol/L NaOH solution for 1 minute. The soaked filter paper was then applied to the center of the cornea for 30 seconds, after which it was removed. The ocular surface and conjunctival sac were subsequently rinsed with physiological saline for 1 minute. Following the modeling procedure, levofloxacin was applied as eye drops to the modeled eye to prevent postoperative infection.

### 2.3 Administration and Dosage

The route of administration was ocular, and treatment commenced the day before modeling. No drug was administered to the blank control group or model control group. In the positive drug group, 50 μL of levofloxacin was applied to the conjunctival sac. In the low concentration group, 50 μL of a 0.03% dihydroquercetin solution was applied, while in the high concentration group, 50 μL of a 0.3% dihydroquercetin solution was applied. All treatments were administered twice daily at 9:00 a.m. and 3:00 p.m.

### 2.4 Measurement of Corneal Neovascularization Area

Fourteen days after treatment, ocular surface inflammation and neovascularization were assessed in each experimental group. The area of neovascularization was imaged, recorded, and subsequently quantified. Data were analyzed statistically using GraphPad Prism 8.0 software.

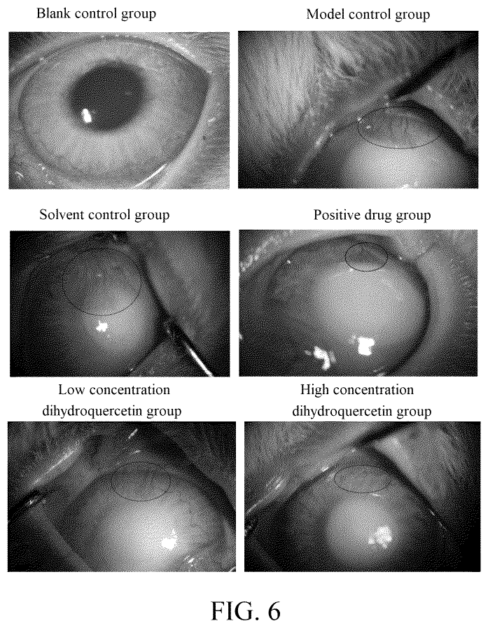

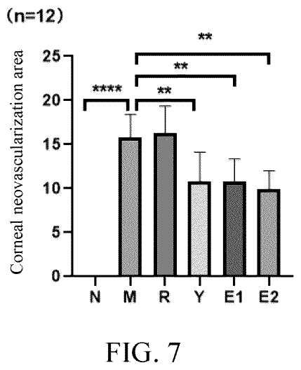

FIG. 6 shows the morphology of corneal neovascularization in New Zealand rabbits from each experimental group observed using a slit lamp for corneal 14 days after treatment, where the black circle represents the area of corneal neovascularization. The blank control group showed a transparent cornea with no signs of neovascularization. The model group and solvent control group showed a large number of dendritic new blood vessels extending from the corneal limbus toward the central cornea, significantly differing from the blank control group. The positive drug group, as well as the low and high concentration dihydroquercetin groups, showed fewer new blood vessels, indicating a significant reduction compared to the model group. The corneal neovascularization area measurements for each group are shown in FIG. 7, where N represents the blank control group; M represents the model control group; R represents the solvent control group; Y represents the positive drug group (levofloxacin), E1 represents the low concentration dihydroquercetin group; and E2 represents the high concentration dihydroquercetin group. In addition, ** represents P<0.01 and **** represents P<0.0001. The corneal neovascularization areas in the model and solvent control groups showed a significant difference compared to the blank control group (P<0.0001). The corneal neovascularization areas in the positive drug control group, low and high concentration dihydroquercetin group were smaller and showed a significant improvement compared to the model group (P<0.01). These results demonstrate that dihydroquercetin can inhibit neovascularization and mitigates damage degree caused by corneal alkali burns.

In summary, dihydroquercetin demonstrates notable efficacy in treating ocular surface diseases. At concentrations of 50-200 μM, dihydroquercetin promotes proliferation in HCE cells, and at concentrations of 25-200 μM, dihydroquercetin offers protection against H₂O₂-induced oxidative stress damage in HCE cells. Treatment with dihydroquercetin results in increased tear secretion and improved tear film stability in mice with dry eye. Furthermore, dihydroquercetin reduces corneal epithelial damage and provides protective effects against corneal alkali burns by inhibiting neovascularization and mitigating associated damage.

The examples provided above are intended to illustrate and elucidate the method and core concept of the present disclosure. It should be noted that those skilled in the art may make various improvements and modifications to the present disclosure without departing from its fundamental spirit. Such improvements and modifications are considered to be within the scope of the claims of the present disclosure.

1. Use of dihydroquercetin in the manufacture of a medicament for the prevention, alleviation and/or treatment of an ocular surface disease.

2. The use according to claim 1, wherein the ocular surface disease is an ocular surface disease caused by corneal epithelial cell damage and/or apoptosis.

3. The use according to claim 2, wherein the corneal epithelial cell damage is oxidative damage.

4. The use according to claim 3, wherein the oxidative damage is caused by hydrogen peroxide.

5. The use according to claim 2, wherein the ocular surface disease is dry eye or a disease related to corneal neovascularization.

6. A dihydroquercetin ophthalmic preparation comprising dihydroquercetin and a solvent.

7. The dihydroquercetin ophthalmic preparation according to claim 6, wherein the dihydroquercetin has a mass concentration of 0.01% to 0.5%.

8. The dihydroquercetin ophthalmic preparation according to claim 6, wherein the dihydroquercetin ophthalmic preparation is in a dosage form selected from the group consisting of eye drops, ophthalmic ointment, periocular and intraocular injection, ophthalmic gel, and liposome.
